(19) United States
(12) Patent Application Publication
(10) Pub. No.: US 2004/0176454 A1
(43) Pub. Date: Sep. 9, 2004

(54) Method of treating an infection

(76) Inventor: Arthur Vanmoor, Boca Raton, FL (US)

Correspondence Address: OTTO S KAUDER, 5382C Venetia Ct., Boynton Beach, FL, 33437, US

(21) Appl. No.: 10/382,927

(22) Filed: Mar. 6, 2003

(51) Int. Cl. A61K 31/198

(52) U.S. Cl. 514/561; 514/563

(57) ABSTRACT

There is disclosed a method of treating an infection in a person in need of such treatment, which comprises the administration to such person of an effective amount of at least one water-soluble aminoacid represented by formula (I)

X—CₙH₂ₙ—CR(NH₂)COOH (I)

wherein X represents a hydrogen atom (H) or a carbamoyl group (CONH₂), R is hydrogen or a methyl (CH₃) group, and n is a whole number from 1 to 4.

### BACKGROUND OF THE INVENTION

1. Field of the Invention

This invention relates to a method of treating a person suffering from an infection with an agent whose effectiveness for this purpose has not previously been recognized.

2. Description of Related Art

As is well known, remedies for infections have been sought for generations by a great variety of methods, and with the increasing application of science some successes have been achieved, particularly in the area of sulfa drugs and antibiotics. However, bacteria, viruses, and other microorganisms believed responsible for the occurrence of infection are known to mutate rapidly, thus giving rise to new strains requiring the development of new remedies. Moreover, the search for such better remedies is enormously costly. For economic reasons, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

In contrast, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds are disclosed in US patent no. as effective against facial pimples; certain water soluble amino carboxylic acid compounds are disclosed in U.S. Pat. No. 5,626,831 as effective against the common cold; certain water soluble amino carboxylic acid compounds are disclosed in U.S. Pat. No. 5,707,967 as effective against headache; certain water soluble amino carboxylic acid compounds are disclosed in U.S. Pat. No. 5,708,029 as effective against elevated blood pressure, and certain water soluble amino carboxylic acid compounds are disclosed in U.S. Pat. No. 5,767,157 as effective against pain in a joint.

U.S. Pat. No. 6,479,547 discloses treating an infection with an aliphatic sulfur compound.

Niibe et al WO 00/37070 discloses carbocysteine 2-amino-3-(S-carboxymethyl)thiopripionic acid as the active ingredient of a drug capable of preventing infectious disease in the pre-infective step of adhesion of bacteria to the respiratory tract.

### SUMMARY OF THE INVENTION

In accordance with this invention, there is provided a method of treating an infection in a person in need thereof, which comprises the administration to such person of an amount of at least one water-soluble aminoacid represented by formula (I) shown below, effective in mitigating the infection.

X—CₙH₂ₙ—CR(NH₂)COOH (I)

In formula (I), X represents a hydrogen atom (H) or a carbamoyl group (CONH₂), R is hydrogen or a methyl (CH₃) group, and n is a whole number from 1 to 4. When n is greater than 1, the alkylene group represented by CₙH₂, can be straight chain or branched.

Without intending to be bound by any theory, the present invention is believed to be beneficial in augmenting the person's innate ability to eliminate toxins from the body and to resist the initiation of the process that makes a person susceptible to infection as well as to slow down, arrest, and even reverse that process. As a result, the incidence of infection and the associated discomfort is diminished, and the quality of life is improved.

In mitigating an infection, mega-nutrient doses of 2 to 20 grams of at least one water-soluble aminoacid represented by formula (I) can be administered from one to five times daily, for a total of 2 to 100 grams per day, until monitoring shows sufficient improvement in the user's condition to permit reduction in dose level and ultimately cessation of the treatment. Daily doses of a water-soluble aminoacid represented by formula (I) in the range from 2 to 40 grams per day are preferred. Such doses can be administered in any convenient manner, as by oral administration in any of the usual dosage forms, such as tablets, capsules, solutions, and dispersions in liquid foods such as soups and fruit juices. Alternatively, there can be given sterile solutions of a water-soluble aminoacid represented by formula (I) by direct injection into the bloodstream of the person to be treated, as well as by rectal suppositories containing a water-soluble aminoacid represented by formula (I).

Infections that can be treated by the method of this invention can be located on the outside of the body or affect internal organs, and include infections caused by bacteria, tropical parasites, or viruses, in particular all forms of dysentery, hepatitis, herpes, pneumonia, syphilis, and typhoid fever.

In formula (I), when n=1 the alkylene group represented by CₙH₂ₙ is a methylene group. When n=2, the alkylene group can be ethylene —CH₂CH₂— or ethylidene —CH(CH₃)—. When n=3 and n=4, the alkylene group represented by CnH₂ₙ can have each of the known isomeric configurations of such straight chain and branched alkylene groups.

The following table contains preferred water-soluble amino acids represented by formula (I) according to the invention.

| Index | Name | X | R | n |
|---|---|---|---|---|
| 1 | 2-aminobutanoic acid | H | H | 2 |
| 2 | 2-amino-2-methylpropanoic acid | H | CH₃ | 1 |
| 3 | 2-amino-3-carbamoylpropanoic acid | CONH₂ | H | 1 |
| 4 | 2-amino-4-carbamoylbutanoic acid | CONH₂ | H | 2 |
| 5 | 2-amino-3-methylpentanoic acid | H | H | 4 |
| 6 | 2-amino-4-methylpentanoic acid | H | H | 4 |
| 7 | 2-amino-3-methylbutanoic acid | H | H | 3 |

### EXAMPLE 1

A 42 year old male neglected a cut on his leg such that gangrene developed. He took 5 grams of a composition of several compounds of formula I four times daily for 16 weeks and observed arrest of the gangrene after two weeks and cure after four weeks.

What is claimed is:

1. A method of treating an infection in a person in need of such treatment, which comprises the administration to such person of an effective amount of at least one water-soluble amino acids represented by formula (I)
X—CₙH₂ₙ—CR(NH₂)COOH (I)
wherein X represents a hydrogen atom (H) or a carbamoyl group (CONH₂), R is hydrogen or a methyl (CH₃) group, and n is a whole number from 1 to 4.

2. The method of claim 1, wherein the aminoacid is administered orally with food.

3. The method of claim 1, wherein the aminoacid is administered by injection into the bloodstream.

4. The method of claim 1, wherein the aminoacid is administered by rectal suppository.

5. The method of claim 1, wherein the effective amount of the aminoacid is administered in one to five daily doses, each dose being in the range of 2 to 20 grain.

6. The method of claim 1, wherein the total of said effective amount of the aminoacid administered daily is in the range of 2 to 100 grams.

7. The method of claim 6, wherein the total of said effective amount of the aminoacid administered daily is in the range of 20 to 50 grams.

8. The method of claim 1, wherein said person experiences relief from the effects of the infection.

9. The method of claim 1 wherein after treatment the infection is not detectable.

10. The method of claim 1 wherein the aminoacid is 2-amino-4-carbamoylbutanoic acid.

11. The method of claim 1 wherein the aminoacid is 2-aminobutanoic acid

12 The method of claim 1 wherein the aminoacid is 2-amino-2-methylpropanoic acid

13 The method of claim 1 wherein the aminoacid is 2-amino-3-carbamoylpropanoic acid

14. The method of claim 1 wherein the aminoacid is 2-amino-3-methylpentanoic acid

15. The method of claim 1 wherein the aminoacid is 2-amino-4-methylpentanoic acid

16. The method of claim 1 wherein the aminoacid is 2-amino-3-methylbutanoic acid

17. The method of claim 1 wherein a plurality of water-soluble aminoacids represented by formula (I) is administered.
